(19) United States
(12) Patent Application Publication
(10) Pub. No.: US 2026/0341145 A1
(43) Pub. Date: Oct. 29, 2026

(54) Surgical Handwashing System

(71) Applicant: OMNImed I, Inc., Tustin, CA (US)

(72) Inventors: Jill Goodwin, Santa Ana, CA (US); Nick Moran, Huntington Beach, CA (US); Robert Brown, Aliso Viejo, CA (US)

(21) Appl. No.: 19/113,635

(22) PCT Filed: Sep. 21, 2023
(86) PCT No.: PCT/US2023/033368

§ 371 Date: Mar. 20, 2025

Related U.S. Application Data
(60) Provisional application No. 63/408,746, filed on Sep. 21, 2022.

(51) Int. Cl. G08B 21/24; A61B 5/11; G06V 10/82; G06V 20/40; G06V 20/52; G06V 40/20; G08B 5/22

(52) U.S. Cl. CPC G08B 21/245; A61B 5/1123; A61B 5/1128; G06V 10/82; G06V 20/44; G06V 20/52; G06V 40/28; G08B 5/22; A61B 2503/20

(57) ABSTRACT

Methods, systems, and apparatus, including medium-encoded computer program products, for monitoring a wash station. A method includes obtaining sensor data from one or more sensors proximate the wash station; identifying, based on the sensor data, a sequence of actions performed by a user at the wash station; comparing the sequence of actions performed by the user at the wash station to a target sequence of actions; determining whether the sequence of actions performed by the user at the wash station satisfies similarity criteria for matching the target sequence of actions; and in response to determining that the sequence of actions performed by the user at the wash station does not satisfy the similarity criteria, generating an alert for presentation on a display device.

### CROSS REFERENCE TO RELATED APPLICATIONS

This application claims the benefit of the U.S. Provisional Patent Application No. 63/408,746 filed Sep. 21, 2022 and entitled “Surgical Handwashing System” which is incorporated herein by reference in its entirety.

### TECHNICAL FIELD

The present disclosure relates to systems and methods for tracking, training, and evaluating actions performed in a medical environment (e.g., a surgery room, a medical clean room). In some examples, the present disclosure is directed to systems and processes for tracking medical personnel while they perform actions within the medical environment, such as handwashing and scrubbing procedures, and for evaluating actions of personnel against best practices in real time and correcting their actions using an expert system as they perform the handwashing and scrubbing procedures.

### BACKGROUND

It is well known that adverse patient outcomes in medical environments have a number of complex causal sources leading to over 250,000 deaths annually in the United States. There exists a cause and effect relationship for adverse patient outcomes between the environmental conditions before and during a surgery and what occurs during and after the surgical procedure performed on a patient. One such relationship exists between inadequate handwashing and scrubbing procedures (i.e., “scrubbing in”) prior to a surgical operation and a resulting sanitation-related adverse outcome, such as a surgical site infection (SSI). A large number of medical complications are related to improper and inadequate handwashing. Although surgical gloves are generally used during operations, these gloves can develop holes, which can provide a route for infection and bioburden. Proper surgical hand scrub procedures can reduce bioburden and risk of infection.

Traditionally, handwashing regulations and standards are not regularly monitored while surgical staff is scrubbing in. A need exists for a system that is specifically focused on monitoring, analyzing, and correcting the handwashing and scrubbing procedures prior to a surgical operation.

### SUMMARY

Embodiments of the present disclosure are directed toward electronic systems that apply sensor fusion to detect, in real time, inconsistencies in handwashing practices, undetectable by human observation, that could result in patient infection during surgical procedures. For example, systems and processes described herein combine multiple sensors and devices, including without limitation, edge computers, three-dimensional stereographic cameras, thermal imaging cameras, temperature sensors, water flow sensors, and microphones; all of which can be augmented by software comprising artificial intelligence (AI) algorithms (e.g., modified “you only look once” (YOLO) models using transfer learning, 1-dimensional and 2-dimensional convolutional neural networks, human pose estimation and human hands and digits position detection via deep neural networks, natural language processing via deep neural networks, multilayer perceptrons, decision trees, and random forest search) and non-AI models (e.g., combination of machine vision image treatment methods, speaker segmentation through audio trace signatures, Fast Fourier Transform, cascade classifier, Mahalanobis distance, connected components labelling algorithm) for tracking, monitoring, analyzing, evaluating, and correcting the handwashing and scrubbing procedures of medical staff prior to surgery, the combination of which can result in detecting and preventing adverse events known to lead to adverse patient outcomes such as SSI. The handwashing system disclosed herein provides a technical advantage over and can supplement human monitoring of handwashing and scrubbing procedures, for example, by providing augmented views (e.g., hand pose models and thermal imaging), sensors (e.g., temperature and spatial telemetry), timers, and alerts based on real-time calculations from the systematic collection of data by the system.

The present disclosure describes systems for monitoring handwashing and scrubbing procedures by medical staff prior to performing surgeries. Systematic collection of handwashing data and sanitation events that can lead to SSI outcomes provides a resource of information that is consistently collected, enabling comparison and analytics that subsequently can be used to conduct epidemiologic assessments. AI-based event data collection and analysis enables the identification of complex latent errors that can be addressed through improved handwashing and scrubbing procedures.

In general, innovative aspects of the subject matter described in this specification can be embodied in methods that include the actions of obtaining sensor data from one or more sensors proximate the wash station; identifying, based on the sensor data, a sequence of actions performed by a user at the wash station; comparing the sequence of actions performed by the user at the wash station to a target sequence of actions; determining whether the sequence of actions performed by the user at the wash station satisfies similarity criteria for matching the target sequence of actions; and in response to determining that the sequence of actions performed by the user at the wash station does not satisfy the similarity criteria, generating an alert for presentation on a display device.

These and other embodiments can include the following features, alone or in any combination. In some implementations, the sensor data includes video data; and identifying, based on the sensor data, the sequence of actions performed by a user at the wash station includes: identifying a part of a human body in the video data; generating, for the part of the human body, a skeletal poise model; and using the skeletal poise model to track movements of the part of the human body in the video data. The sequence of actions performed by the user at the wash station includes a sequence of movements of the part of the human body.

In some implementations, identifying, based on the sensor data, the sequence of actions performed by the user at the wash station includes determining, for each action of the sequence of actions, a duration of the action, and comparing the sequence of actions performed by the user at the wash station to a target sequence of actions includes comparing the duration of each action of the sequence actions performed by the user at the wash station to a duration of each action of the target sequence of actions.

In some implementations, determining that the sequence of actions performed by the user at the wash station does not satisfy similarity criteria for matching the target sequence of actions includes determining that a duration of at least one action of the sequence of actions performed by the user at the wash station is less than a duration of a corresponding action of the target sequence of actions.

In some implementations, identifying, based on the sensor data, the sequence of actions performed by the user at the wash station includes determining, based on the sensor data, an ordering of the sequence of actions performed, and comparing the sequence of actions performed by the user at the wash station to a target sequence of actions includes comparing the ordering of the sequence actions to an ordering of the target sequence of actions.

In some implementations, determining that the sequence of actions performed by the user at the wash station does not satisfy similarity criteria for matching the target sequence of actions includes determining that the ordering of the sequence of actions performed by the user at the wash station is different from the ordering of the target sequence of actions.

In some implementations, the target sequence of actions includes washing actions executed on a user's hands and forearms, including one or more of: removing jewelry; rinsing the user's hands and forearms with water; cleaning fingernails with a nail pick; scrubbing the user's hands and forearms with an iodine sponge; passing the user's hands and forearms through running water in a forward direction; washing the user's hands and forearms in a direction from the fingertips towards the elbow; and drying the user's hands and forearms with a towel.

In some implementations, the target sequence of actions includes maintaining positions of parts of a user's body, including one or more of: maintaining the user's elbows bent at or near a right angle; and maintaining the user's hands above the level of the elbows.

In some implementations, the target sequence of actions includes introducing one or more objects into the wash station, and comparing the sequence of actions performed by the user at the wash station to a target sequence of actions includes comparing an object introduced into the wash station to the one or more objects introduced in the target sequence of actions.

In some implementations, determining that the sequence of actions performed by the user at the wash station does not satisfy similarity criteria for matching the target sequence of actions includes determining that the object introduced into the wash station does not match the one or more objects introduced in the target sequence of actions.

In some implementations, the target sequence of actions includes washing a part of a user's body using water having a temperature within a designated range of temperatures, and comparing the sequence of actions performed by the user at the wash station to a target sequence of actions includes comparing the temperature of water used to wash the part of the user's body to the designated range of temperatures.

In some implementations, determining that the sequence of actions performed by the user at the wash station does not satisfy similarity criteria for matching the target sequence of actions includes determining that the temperature of the water used to wash the part of the user's body is not within the designated range of temperatures.

In some implementations, the sensor data includes video images from at least one of a thermal imaging camera or a stereographic camera.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1 is a block diagram representing exemplary features of a medical process monitoring or training system according to some embodiments of the present disclosure.

FIG. 2 is floor diagram illustrating an exemplary arrangement of sensors and system components of a medical process monitoring or training system according to some embodiments of the present disclosure.

FIG. 3 depicts a side view of a handwashing system according to some embodiments of the present disclosure.

FIGS. 4-7 depict screenshots of exemplary graphical user interface output from a handwashing system according to some embodiments of the present disclosure.

FIG. 8 depicts a flowchart of an example process for monitoring surgical handwashing.

FIG. 9 depicts a block diagram of a computer system that may be applied to any of the computer-implemented methods and other techniques described herein.

Like reference numbers and designations in the various drawings indicate like elements.

### DETAILED DESCRIPTION

Embodiments of the present disclosure are directed at capturing inadequate handwashing and scrubbing procedures having potential causal links to adverse patient outcomes, such as SSI, through the use of a combination of many sensory device signals augmented by artificial intelligence algorithms (e.g., modified YOLO models using transfer learning, 1-dimensional and 2-dimensional convolutional neural networks, human pose estimation and human hands and digits position detection via deep neural networks, natural language processing via deep neural networks, multilayer perceptrons, decision trees, and random forest search) and non-AI models (e.g., combination of machine vision image treatment methods, speaker segmentation through audio trace signatures, Fast Fourier Transform, cascade classifier, mahalanobis distance, connected components labeling algorithm) to detect, analyze and recommend corrective actions in real-time or near real-time prior to the surgical operation.

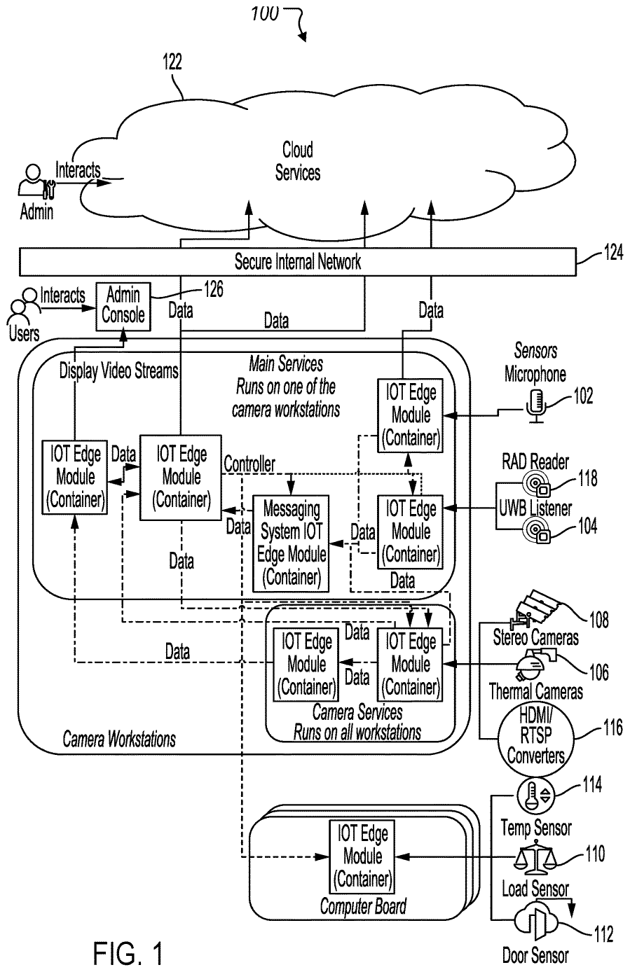

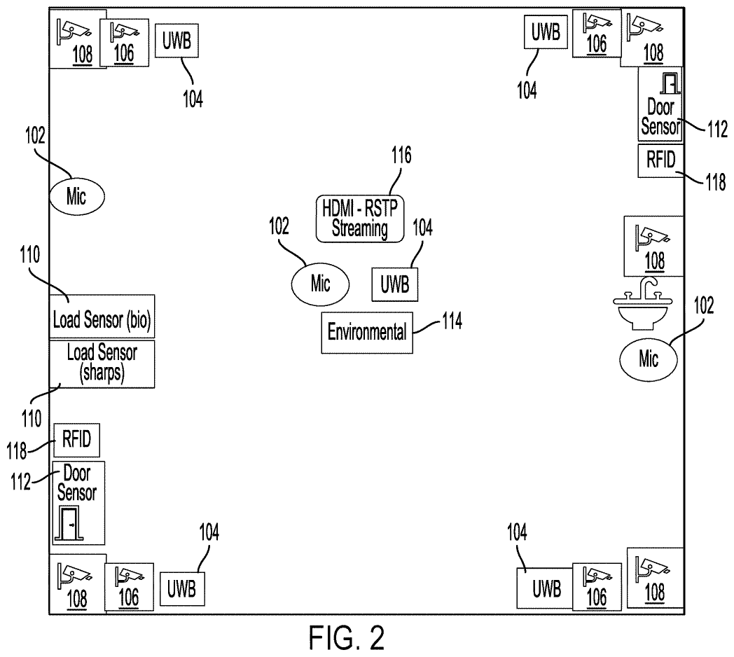

Referring to FIGS. 1 and 2, an example system 100 for medical process monitoring or training includes a combination of sensors, devices, and other elements, such as microphone(s) 102, real-time locating system (RTLS) tracking sensor receivers and sensors (e.g., ultra-wide band (UWB) anchors and sensors(s)) 104, thermal imaging camera(s) 106, stereographic camera(s) 108, load sensor(s) 110, door sensor(s) 112, environmental sensor(s) 114 that can sense temperature, pressure, humidity, static pressure, dynamic pressure, particles (e.g., between 0.5 μm and 1.0 μm), and carbon-dioxide, high-definition multimedia interface (HDMI) rapid spanning tree protocol (RSTP) wireless streaming device(s) 116 to send and/or receive medical imaging data for display on one or more display devices, and/or radio-frequency identification (RFID) readers 118, all within an AI-based data-driven behavior learning approach along with (optionally) cloud-based and edge-based computing device(s) (e.g., IOT edge modules, messaging systems, sensor APIs, camera APIs, and media servers) that collect and logically assemble cause and effect evidence to ultimately explain adverse patient outcomes and prevent them and/or alarm on adverse contributors in real-time while a surgery is ongoing from an admin console 126, which can be is secured by an internal network 124. Additional sensors are also possible, including for example, air flow sensors and air particle count sensors.

Some embodiments provide a system that can combine its various sensors' data through an aggregator subsystem and apply AI algorithms through a processor to extract and accumulate the following information in real-time during surgical procedures in an operating room, medical clean room, or training room.

Example operations of the system include detecting specific medical instrument drop events through the combination of feeding and processing of video data streams from cameras 106, 108 to clustering and machine vision algorithms (e.g., combination of continuous video frame differencing and background subtraction, video stream object movement noise filtering with human pose estimation via deep neural networks, video stream object movement noise filtering with density-based spatial clustering of applications with noise (DBSCAN), Gaussian mixture-based background/foreground segmentation for video stream object drop detection in front of other moving objects, Farneback optical flow for video stream movement speed segregation in special cases, image color detection, image segmentation, pixel clustering, pixel thresholding, morphology operations using erode and dilate and/or find contour, video frame processing using cascade filters, windowing. skipping frames, and down sampling, image enhancement using Gaussian blur, kernelling, Laplacian filter, change of color space, image feature detection using Hough Transform, measurement using Euclidean distance and mahalanobis distance, stereovision, histogram, and watershed), and feeding and processing high frequency audio data from microphones 102 to a separate instrument audio signal signature for classification.

Example operations of the system include performing automated medical instrument usage and count through feeding and processing of the video data streams from cameras 106, 108 to machine vision background subtraction, color filtering, and connected components labeling algorithms.

Example operations of the system include classifying medical instruments through feeding and processing of the video data streams from cameras 106, 108 to a trained AI machine vision (e.g., via a modified YOLO model architecture using transfer learning and deep convolutional neural networks) capable of locating and identifying instruments within the video stream.

Example operations of the system include tracking human body motion through the room by feeding and processing of the video data streams from cameras 106, 108 to an AI model via a deep neural network that can associate and estimate a human skeletal pose model and location within the video data stream for gathering accurate human body motion and, in a similar process, also track human hand and finger movement.

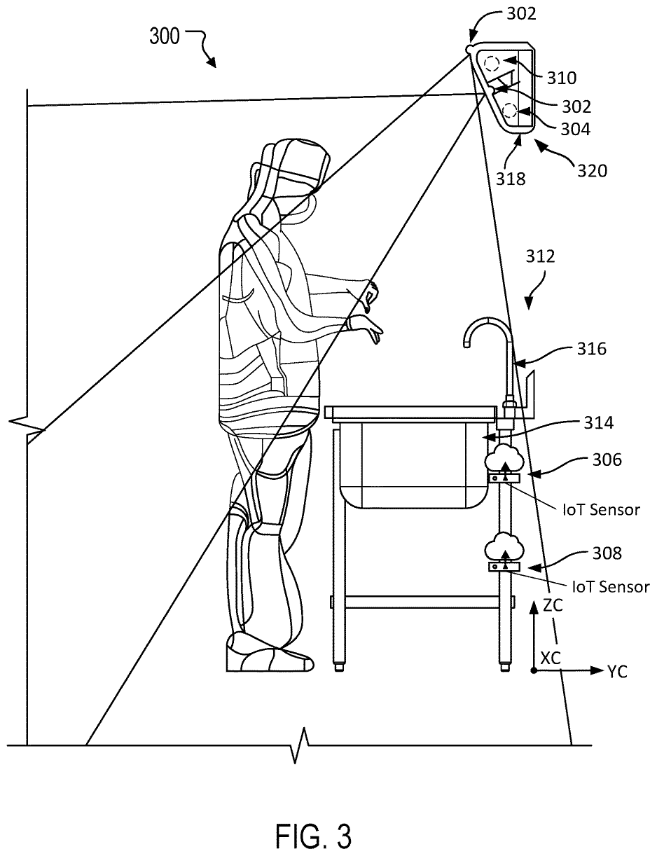

Example operations of the system include validating whether the pre-surgery handwashing procedure is satisfactory through feeding and processing of the video data streams from cameras 106, 108 and validating spatial movement and water flow through audio processing (e.g., via 1-dimensional and 2-dimensional convolutional neural networks or Fast Fourier Transform with spectral filtering) from microphone 102. Monitoring of pre-surgery handwashing procedures is described in greater detail with respect to FIGS. 3 to 8.

Example operations of the system include identifying virtual zones (which may be two-dimensional or three-dimensional), such as sterile zones around the surgical table and other critical zones in the room, augmented and overlaid onto the video data streams from cameras 106, 108 using, e.g., cuboid algorithms to identify breach in zone from the AI-based location estimation of the human skeletal pose model in the room.

Example operations of the system include identifying hot spots through feeding and processing of thermal imaging data streams from thermal imaging cameras 106, 108.

Example operations of the system include detecting and tracking potential infection inception areas in the room by linking the location of the hot spots to one or more zones from the underlying stereographic video data streams.

Example operations of the system include detecting and tracking breach of human movement into zones by comparing the tracked human body motion in relation to the augmented, zones to evaluate if the human skeletal pose model intersects with one of the zones.

Example operations of the system include measuring the weight of biohazard waste and general surgical procedure waste using load sensors 110.

Example operations of the system include detecting and tracking authorized door entries and exits by feeding and processing of the video data streams from cameras 106, 108 to an AI via deep neural network and associate the human skeleton pose model and location within the video data stream for gathering accurate human body motion colliding with door zones defined in the system and, in a similar way, track unauthorized door entries and exits.

Example operations of the system include detecting and tracking door open and door close events based on a door signal from door sensors 112. Example operations of the system include tracking particle count using air particle sensors. Example operations of the system include tracking air refresh cycle using airflow sensors. Example operations of the system include transcribing speech to text by feeding audio signals from microphones 102 to an AI natural language processing model and audio voice transcription via deep neural networks and text enhancements using transformer deep neural networks to achieve textual transcriptions and perform speaker identification using an ensemble of classifier models.

Systematic collection of all potential adverse events during training and during actual surgical medical procedures provides a resource of information consistently collected enabling comparison and analytics that can be used at a later date to conduct epidemiologic assessments and then prevent adverse events from happening in the first place using real-time notifications and feedback.

In one or more embodiments, a system can include one or more of the sensors and/or devices discussed above, all connected through a network apparatus which itself can be connected to a cloud computer service 122. The system, through its multiple sensors, can have an aggregator subsystem combining the sensors inputs to feed them to at least one AI algorithm. The system can have the ability to define virtual, three-dimensional zones such as room door entries, room door exits, sterile surgical zones, and other three-dimensional zones of importance for analytics; all of which are then leveraged by the overall system's algorithms. The tracking of human body and hand motion is computed by feeding the three-dimensional stereographic data to an AI model. The resulting positions in three-dimensions in the room are then compared to virtual zones, such as a sterile surgical zone, for computing real-time breaching of the zones. In a similar way, a human body motion entering a door zone or exiting a door zone generates an event tracking these entries and exits. In combining virtual zones and sensor data fusion, the system is configured for tracking, detecting, and analyzing the potential adverse events happening within the entire surgical room or medical clean room through the tracking of at least the biometric movement of the users, users' hands, medical instruments, and medical devices being used to perform a medical procedure by at least one user over at least one body form within a surgical training space or a real surgical space. At least one central apparatus can be mounted in the medical training or surgical space. An example central apparatus 320 is shown in FIG. 3. The apparatus 320 can also be connected to one or more stereographic cameras 106, 108, each with a thermal imaging camera 106, that are able to capture the user's biometric movement in the training or surgical space as well as thermal and environmental conditions of objects and people in the room, as one or more tasks are performed by at least one user. Each stereographic camera 108 and thermal imaging camera 106 can be connected to a dedicated edge microprocessor to capture the live video, live audio, live thermal data, and the spatial data generated by each camera. The system can use a dedicated pose detection engine on the edge microprocessor, which can be connected to a dedicated computer through an internal network connection, to compute and detect breach events. The computer can be operable through an administrative interface, and/or the cloud administrative interface, to invoke the recording and storage of the live video, audio, sensor telemetry, and spatial data captured from a starting capture point of the procedure, and concluding with an end capture point of the procedure over a variable time span determined by the user or automatically determined from the captured data.

In some examples, the sensors can be mounted in particular arrangement within a medical procedure room or medical clean room. For example, a stereographic camera 108, a thermal imaging camera 106, a tracking sensor receiver 104, and a microphone 102 can be co-located in each corner of the room and directed towards the center of the room. In some examples, a module of sensors is positioned in each corner of the room. The module can be a housing that contains a stereographic camera 108, thermal imaging camera 106, a tracking sensor receiver 104, and a microphone 102. In some examples, an additional stereographic camera 108 is located in the center of the room. For example, the additional camera can be mounted to the ceiling of the room and directed downwards at an operating table. In some examples, a module of sensors is mounted from the ceiling above the operating table and directed towards the operating table.

In some examples, coordinate systems for other sensors are calibrated to correspond with a coordinate system of the stereographic cameras 106, 108. For example, a coordinate system for RTLS tracking sensors can be calibrated to correspond with the coordinate system of the stereographic cameras 106, 108. An exemplary calibration process includes removing all or most equipment from a room, if needed. Placing an ArUco marker at the center of the room as a reference point for each camera. Calibrating a coordinate system based on video feeds from the stereographic cameras mounted in the corners of the room based on the ArUco marker at the center of the room. For instance, stereographic camera coordinate system can be calibrated based on the location and orientation of the ArUco marker. ArUco markers can also be placed on a plurality of RTLS sensors distributed around the room. The system can determine the position of each sensor relative to each RTLS receiver, and determine the location of each sensor within the video feeds of the cameras and within the video based coordinate system. The system can correlate the RTLS position data with the video position data based on the ArUco makers to calibrate the RTLS sensor system with the video based coordinate system of the cameras.

Referring to FIG. 3, a system 300 can include a combination of sensors, devices, and other elements, such as stereographic camera(s) 302, thermal imaging camera(s) 304, temperature sensor(s) 306, water flow sensor(s) 308, and microphone(s) 310, along with (optionally) cloud-based and edge-based computing device(s) (e.g., internet of things (“IOT”) edge modules, messaging systems, sensor application programming interfaces (“APIs”), camera APIs, and media servers) that collect and logically assemble cause and effect evidence to ultimately explain and prevent adverse events such as SSI. The system 300 can be a subsystem of the system 100.

Some embodiments provide a system that can combine its various sensor devices data through an aggregator subsystem and apply AI algorithms through a processor to extract and accumulate information in real-time during the handwashing and scrubbing process. For example, the system can validate whether the pre-surgery handwashing procedure is satisfactory through feeding and processing of the video data streams received from stereographic cameras 302 and validating spatial movement and water flow through audio processing (e.g., via 1-dimensional and 2-dimensional convolutional neural networks or Fast Fourier Transform with spectral filtering) data received from microphones 310.

Some embodiments provide a system that can combine its various sensor devices' data through an aggregator subsystem and apply AI algorithms through a processor to extract and accumulate the following information in real-time during handwashing and scrubbing procedures.

The system 300 can detect, identify, and track objects within a wash station 312 that includes a sink 314 and a faucet 316. For example, the system 300 can identify acceptable objects (e.g., objects that are expected to be involved in the handwashing process, such as a nail pick or iodine sponge) and unacceptable objects (e.g., objects that are prohibited from or not expected to be involved in the handwashing process, such as jewelry accidently left on the hand or wrist), through feeding and processing of the video data streams from stereographic cameras 302 to a trained AI machine vision (e.g., via a modified YOLO model architecture using transfer learning and deep convolutional neural networks) capable of locating and identifying objects within the video stream.

The system 300 can track human hand motion within the wash station 312 by feeding and processing of the video data streams from cameras 302, 304 to an AI model via a deep neural network that can associate and estimate a human hand pose model and location within the video data stream for gathering accurate human hand motion to analyze the positioning of the hands, identify errors in real time, and provide corrective feedback.

The system 300 can include one or more machine learning models that are trained to identify washing actions performed at the wash station 312. The machine learning models can be trained to identify correct and incorrect washing routines. In some examples, the machine learning models are trained by receiving, as training input, sensor data captured from washing stations in which correct washing routines are performed. For example, a machine learning model can be trained by receiving, as training input, video images of correct washing routines that are labeled as “correct,” and video images of incorrect washing routines that are labeled as “incorrect.” The trained machine learning model can then receive, as input, video images of a washing routine performed at the wash station 312, and can determine whether the washing routine is correct or incorrect.

In some examples, the machine learning models can be trained to identify specific actions performed at the wash station 312. For example, a machine learning model can be trained by receiving, as training input, video images of scrubbing actions that are labeled as “scrubbing,” video images of rinsing actions labeled as “rinsing,” and video images of nail cleaning actions labeled as “nail cleaning.” The trained machine learning model can then receive, as input, video images of washing actions performed at the wash station 312, and can label the washing actions. The system can then compare the labeled washing actions to target washing actions (e.g., washing actions performed in correct washing routines).

The system 300 can validate whether the pre-surgery handwashing procedure is satisfactory through feeding and processing of the video data streams from stereographic cameras 302 and validating spatial movement and water flow through audio processing (e.g., via 1-dimensional and 2-dimensional convolutional neural networks or Fast Fourier Transform with spectral filtering) of audio data from microphones 310. The system 300 can identify virtual zones for the wash station 312 and the sink 314.

The system 300 can time stamp the duration of each step of the surgical scrub, including recording the time stamp of each detected object entering the wash station.

The system 300 can verify acceptable water temperature (e.g., pursuant to standards set by the Occupational Safety and Health Administration (“OSHA”)) through feeding and processing of temperature data streams from temperature sensors 306.

In one or more embodiments, a system can include one or more of the sensors and/or devices discussed above, all connected through a network apparatus which itself can be connected to a cloud computer service (e.g., cloud services 122). The system, through its multiple sensors, can have an aggregator subsystem combining the sensor devices' inputs to feed them to at least one AI algorithm.

As shown in FIG. 3, stereographic cameras 302 can capture video streams of a user (e.g., a surgical staff member) and the wash station 312 with the sink 314 and the faucet 316. As shown, a central apparatus 320 can be positioned above the wash station 312. The central apparatus 320 includes a housing 318 that encloses or supports devices such as stereographic cameras 302, thermal imaging cameras 304, and microphones 310. The system can process video from stereographic cameras 302 to identify various indications relevant to proper sanitation and handwashing. For example, stereographic cameras 302 can allow the system to identify unacceptable objects in the wash station 312 such as rings on the user's hand and bracelets or watches on the user's wrist. Stereographic cameras 302 can also allow the system to identify sanitation concerns such as residual water dripping into the sink 314, whether it is the user's first case of the day, cuts or wounds on the user's skin, dirt or soil on the user's fingertips, the user's fingernails extending beyond the fingertips, chips or scratches in the user's nail polish, acrylic or artificial fingernails, foreign bodies on the user's fingernails (e.g., gems, rhinestones), the user's hair not tucked into a surgical head covering, the user's ears not covered by a surgical head covering, and neck jewelry not tucked into the user's scrubs, in addition to proper use of personal protection equipment, such as eye protection and surgical masks, including whether the mask covers the user's nose, mouth, and facial hair. The system can also process video from stereographic cameras 302 to identify non-sterile surfaces and whether the user's hands, forearms, or elbows have contacted any non-sterile surfaces.

Stereographic cameras 302 can allow the system to identify acceptable objects as well, such as an iodine (e.g., povidone-iodine) sponge packet, and detect the opening and placement of the iodine sponge packet and whether the opening in the iodine sponge packet is facing up toward the ceiling. Additionally, stereographic cameras 302 can identify the location of the iodine sponge once it has been removed from the packet and distinguish between the abrasive side and the non-abrasive side of the sponge. Other acceptable objects that the system can identify include a nail pick and a trash can, where stereographic cameras 302 can identify whether the nail pick, iodine sponge, and iodine sponge packet have been thrown away in the trash can.

Handwashing techniques that the system can identify based on video from stereographic cameras 302 include counting back and forth stroke motions of the iodine sponge, whether the iodine sponge is under running water, whether the user squeezes the iodine sponge, whether iodine comes out of the iodine sponge, whether the user rinses both hands and both forearms under water, whether the user rinses with the fingertips together and side by side, whether the user performs a pre-wash with soap from a soap dispenser near the sink 314, whether the user performs a scrub after the pre-wash with the iodine sponge, whether the nail pick is used under running water, whether the nail pick is used on each nail of both of the user's hands, and whether the user washes from the top of the thumb to the wrist. The system can also identify the palm side and dorsal side of the user's hand, the sides of each finger, and the thirds of the forearm (wrist to midarm, midarm to bottom of elbow, and bottom of elbow to two inches above the elbow), including the top, bottom, and sides of each third of the forearm, and based on identifying the user's hands and arms, whether the user has rinsed and scrubbed in the correct order of (i) fingertips, (ii) hand, (iii) forearm, and (iv) elbow and in the correct hand and arm positioning where the hands and arms are moving in one forward direction through the water and not back and forth, the elbows are bent at or near right angles, and the hands are above the level of the elbows. The system can time stamp the duration of each step of the surgical scrub (e.g., handwashing initiated by entry into the wash station 312, pre-rinse, nail cleaning, hand and arm scrub, end wash by leaving the wash station 312), including recording the time stamp of any sanitation alert, such as a detected unacceptable object entering wash station 312. After the user performs a first rinse, the system can identify whether the user repeats the process with an additional rinse.

After the user completes the handwashing process, the system can identify, based on video from stereographic cameras 302, whether the user incorrectly shakes or waves the hands to dry or correctly uses a sterile towel for drying, and also, whether the user exits the wash station 312 backwards.

Based on temperature sensors 306, the system can detect the temperature of running water from faucet 316, for example, identifying whether the water is within an acceptable range from around 105 ° F. and 120° F. (40.6° C. to 48.9° C.). Thermal imaging cameras 304 can allow the system to identify hot spots on the user's arms and hands. The system can also detect whether water is running from faucet 316 based on water flow sensors 308.

Microphones 310 allow the system to perform natural language processing (NLP), speech to text, detect object drops (such as the user dropping the nail pick or iodine sponge), and automated actions, such as touchless sensor activation in faucets 316.

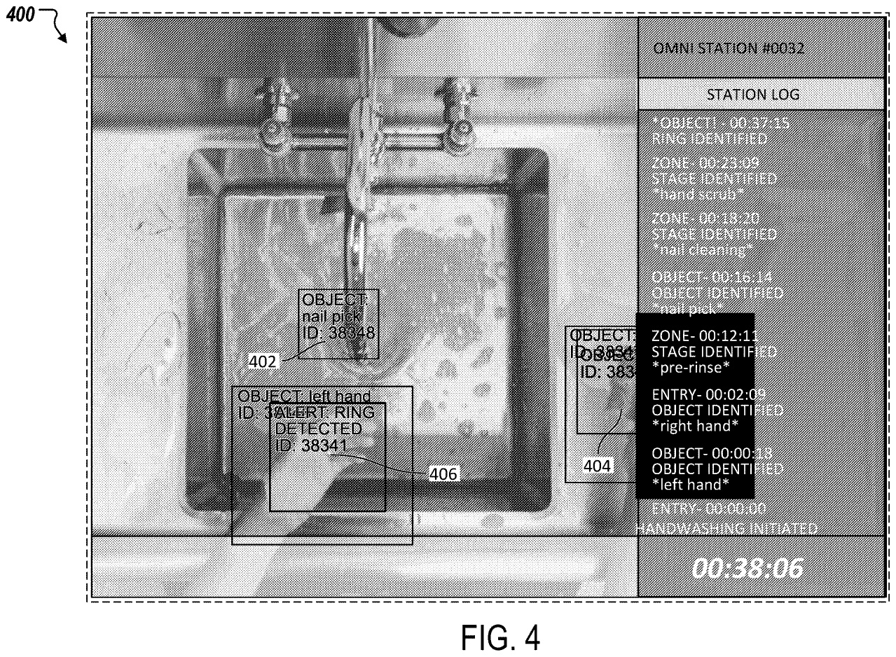

As shown in FIG. 4, a user interface 400 can display video of the wash station 312, and can display information related to identification and tracking of objects within wash station 312. Examples of objects that the system can identify include, without limitation, acceptable objects, such as a nail pick 402 or iodine sponge 404, and unacceptable objects, such as jewelry 406 accidently left on the hand or wrist. The user interface 400 can color code objects, for example, by showing acceptable objects in green within the user interface 400 and showing unacceptable objects in red the user interface 400. The system can classify these and other objects within the wash station 312 through feeding and processing of the video data streams from stereographic cameras 302 to a trained AI machine vision (e.g., via a modified YOLO model architecture using transfer learning and deep convolutional neural networks) capable of locating and identifying objects within the video stream generated by stereographic cameras 302. The user interface 400 can initiate and display a sanitation alert when the system detects an unacceptable object in the wash station 312.

In addition, the system can identify, using video of the wash station 312 generated by stereographic cameras 302, actions being performed at the wash station 312. For example, the system can identify, based on processing the video data streams from stereographic cameras 302 using a trained AI machine vision, actions corresponding to various stages of a surgical handwashing routine. Stages can include, for example, removal of jewelry, initiation of hand washing, pre-rinse, nail cleaning, left hand scrub, right hand scrub, left forearm scrub, right forearm scrub, and drying.

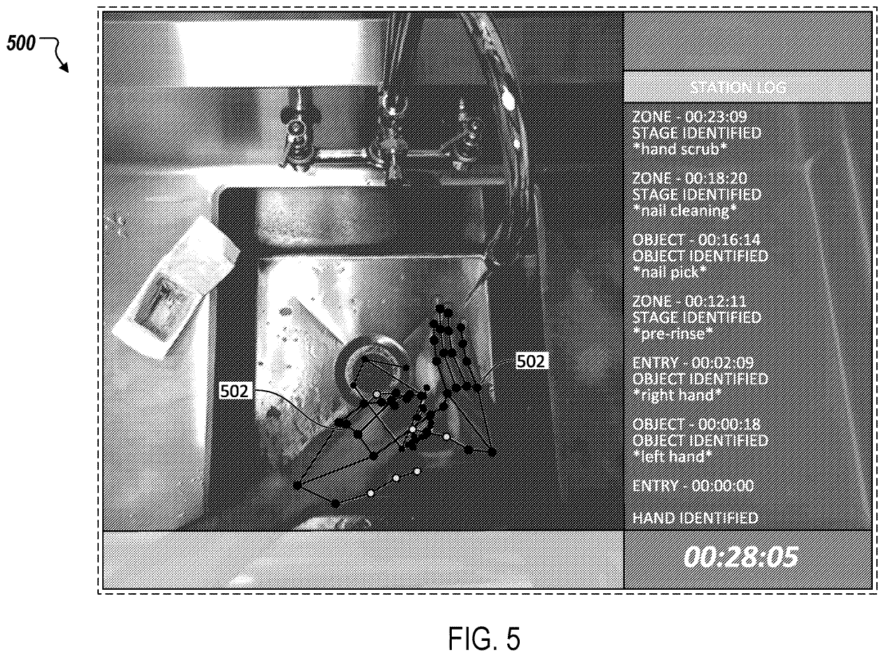

As shown in FIG. 5, the system 300 can generate and a user interface 500 can display a hand pose model 502, which identifies hand skeletal points and displays a skeletal overlay on the video data stream from stereographic cameras 302. The system can track human hand motion in the wash station 312 by feeding and processing of the video data streams from stereographic cameras 302 to an AI model via a deep neural network that can locate, associate, and estimate hand pose model 502 within the video data stream for gathering accurate human hand motion to analyze the positioning of the hands. The system can compare the detected positioning of the hands to target hand positions (e.g., hand positions corresponding to adequate handwashing techniques). The system can determine, based on the positioning of the hands, actions being performed by the user. The system can compare the actions performed by the user to target actions representing correct or appropriate washing techniques. Target actions can include, for example, scrubbing between fingers, scrubbing in the direction of fingertips to elbows, and cleaning underneath fingernails).

The system can identify errors in the user's hand washing techniques in real time or near real time and can provide corrective feedback. For example, the system can determine, in real-time, that the user has an incorrect hand position, and can generate an alert for presentation through the user interface 500 indicating the incorrect hand position. In some examples, the system can determine that the user has skipped a step of a surgical handwashing routine, and can generate an alert for presentation through the user interface 500 indicating that the user skipped the step of the surgical handwashing routine. In some examples, the system can determine that the user performed steps out of order. For example, the system can determine that the user washed the wrists before washing the palms, and that the target order of steps is to wash the palms before washing the wrists. In response, the system can generate an alert for presentation through the user interface 500 indicating that the user performed the hand washing steps out of order.

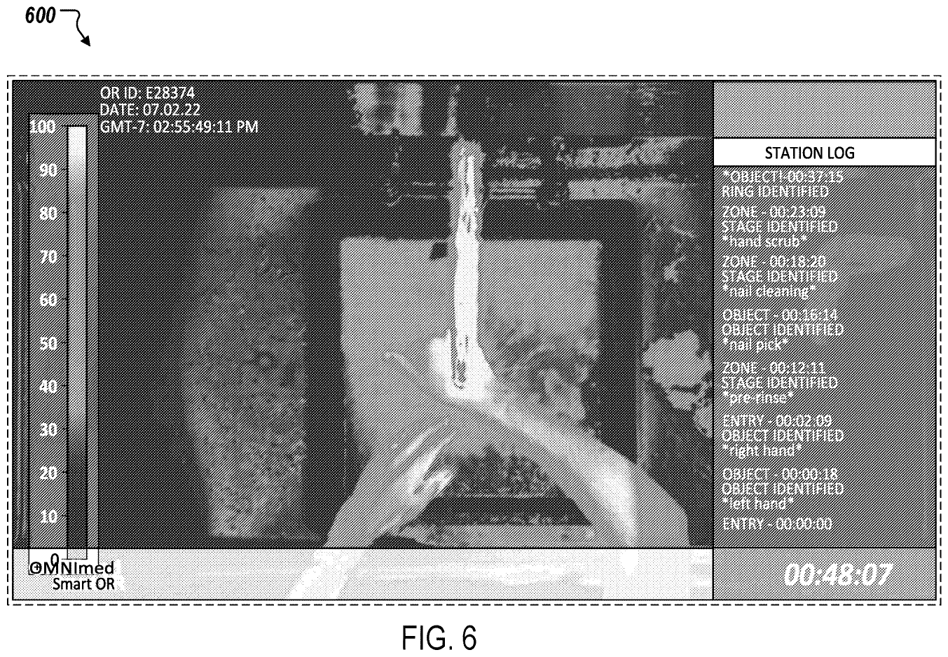

As shown in FIG. 6, the user interface 600 can display thermal imaging from thermal imaging cameras 304 and infrared technologies to show areas with increased heat that can relate to or cause infection inception areas, which can contribute to SSI. The system can also track potential infection inception areas in the wash station 312 by identifying hot spots on a user's hands and arms. The system can determine, based on data generated by the thermal imaging cameras 304, a temperature of water used to wash the user's hands and arms, and can verify that the temperature of the water is within an allowed range of temperatures. In response to determining that the temperature of water is not within the allowed range of temperatures, the system can generate an alert for presentation through the user interface 600 indicating that the temperature of water is not within the allowed range of temperatures.

In some examples, the system can determine, using sensor data from water flow sensors 308, microphones 310, stereographic cameras 302, or any combination of these, that water is flowing from the faucet 316. In some cases, the system can determine, using the sensor data, that a flow rate of water flowing from the faucet 316 is within an acceptable range of flow rates.

In some examples, the system can determine, using sensor data from water flow sensors 308, microphones 310, stereographic cameras 302, or any combination of these, that no water is flowing from the faucet 316, or that water flowing from the faucet is not within an acceptable range of flow rates. In response to determining that no water is flowing from the faucet 316, or that water flowing from the faucet is not within an acceptable range of flow rates, the system can perform one or more actions such as generating an alert.

In some examples, the system can determine, using thermal imaging cameras 304, temperature sensors 306, or both, that the temperature of the water is within an acceptable range.

In some examples, the system can determine, using thermal imaging cameras 304, temperature sensors 306, or both, that the temperature of the water is not within an acceptable range. In response to determining that the temperature of the water is not within an acceptable range, the system can perform one or more actions such as generating an alert.

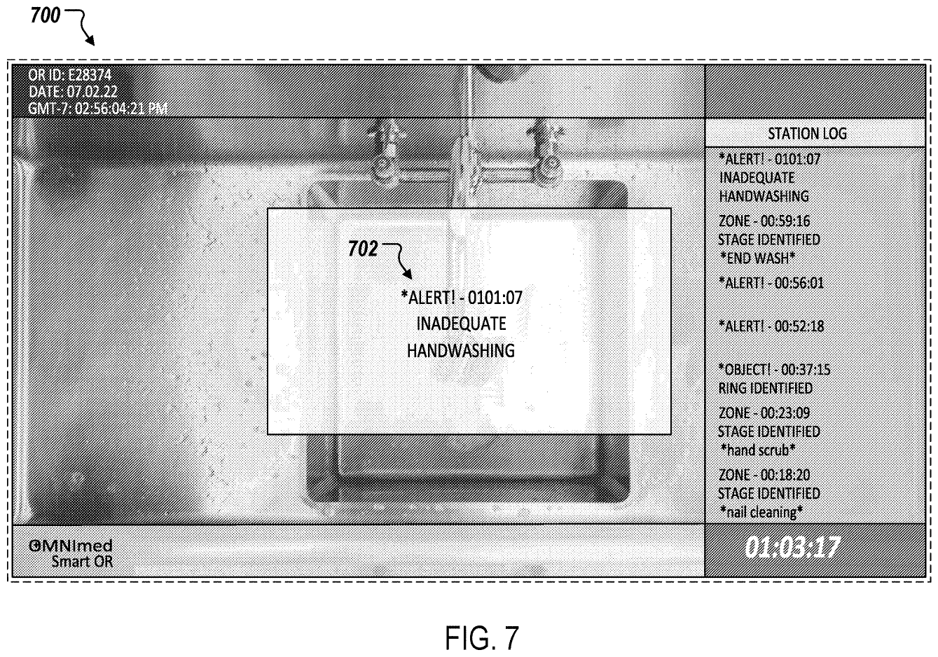

As shown in FIG. 7, the user interface 700 can display an alert 702 (e.g., “inadequate handwashing”) when sanitation has been compromised by a sanitation event, such as “ring identified.” In some examples, in addition to or instead of displaying a textual alert 702, the system can generate an audible alert such as an alarm, can activate a visual alert such as a flashing light, can transmit a notification to another computing device, or any combination thereof.

The system can define virtual zones such as for the wash station 312 and the sink 314. The system can track objects entering and exiting the virtual zones, and can track actions performed in each of the virtual zones. For example, the system can track when human hands are inserted into a virtual zone corresponding to the sink 314, and can track motion of the human hands while the hands are in the virtual zone corresponding to the sink to evaluate effectiveness of handwashing actions. The tracking of human hand motion is computed by feeding the three-dimensional stereographic data to an AI model. In combining virtual zones and sensor data fusion, the system is configured for tracking, detecting, and analyzing the sanitation events happening within wash station 312 through the tracking of at least the biometric movement of the users' hands and other objects in wash station 312.

Referring to FIG. 3, at least one central apparatus 320 can be mounted near wash station 312. The apparatus 320 can also be connected to one or more stereographic cameras 302, each with a thermal imaging camera 304, that are able to capture the user's biometric movement in the training or surgical space as well as thermal and environmental conditions of objects and people in the room, as one or more tasks are performed by at least one user. Each stereographic camera 302 and thermal imaging camera 304 can be connected to a dedicated edge microprocessor to capture the live video, live audio, live thermal data, and the spatial data generated by each camera. The system can use a dedicated pose detection engine on the edge microprocessor, which can be connected to a dedicated computer through an internal network connection, to compute and detect sanitation events. The computer can be operable through an administrative interface, and/or the cloud administrative interface, to invoke the recording and storage of the live video, audio, sensor telemetry, and spatial data captured from a starting capture point of the procedure, and concluding with an end capture point of the procedure over a variable time span determined by the user or automatically determined from the captured data.

Each handwashing session is automatically tracked and recorded by the system. Data related to the handwashing session generated by the sensors can be accessed using a user interface. A handwashing session can include a user approaching the wash station 312, performing handwashing procedures, and departing from the wash station 312. According to an embodiment of the present disclosure, the user interface can provide a playback mode to allow a user to rewind and pause the session while still in progress. The user interface can also provide an archive mode to allow a user to review a session that has completed.

According to another aspect of the present disclosure, a server can be connected to a cloud service (e.g., cloud services 122) that analyzes the combined video, audio, sensor telemetry, and spatial data stream to generate augmented feedback in real time to the user at the wash station 312 or users in the room. The cloud service can also include an analysis engine to analyze the data and develop data modeling utilizing a Naïve Bayes model, enabling efficient, contextual data analysis using classification algorithms. The cloud service can also be operable to generate handwashing quality metrics for the users based at least on the video from stereographic cameras 302, audio signals from microphones 310, thermal imaging data from the thermal imaging camera 304, temperature data from the temperature sensor 306, sensor telemetry, spatial data stream analysis of the biometric movement of the user, user's hands, object (e.g., nail pick and sponge) movement, sensor telemetry, and spatial data.

According to another aspect of the present disclosure, the cloud service 122 can also include a learning engine to develop augmented feedback created by the analysis engine to be delivered through the network to a display device, such as a tablet screen mounted to the wash station, which can display in user interface and augmented feedback that is indicative of the quality of the real-time analyzed handwashing quality of the one or more users based at least on the position data during the handwashing procedure. Once cause and effect of SSI outcomes are learned by one or more artificial intelligence engines, such as those discussed above, the feedback may include recommendations in near real-time for the user to take corrective actions to avoid SSI outcomes.

According to another aspect of the present disclosure, the cloud service 122 can utilize a computer administrative interface or the cloud administrative interface (or admin interface), enabling the administrative user (also referred to herein as admin user) to oversee multiple users and capture and review video, audio, sensor telemetry, and spatial data captured and stored within the analysis engine and the learning engine to provide metrics indicative of the quality of handwashing of the one or more users based at least on the position data and related sanitation event data collected by the system in real-time or in play-back mode.

According to another aspect of the present disclosure, the cloud service 122 can utilize a content management engine that can be accessed through the admin interface on the computer, enabling the user to analyze and annotate stored video, audio, sensor telemetry, and spatial data captured by the system held within the learning engine, giving the user admin the ability to generate customized real time evaluation and augmented feedback to the user during the procedure.

The cloud service 122 can utilize a content management engine that can be accessed through the admin console, enabling the user to analyze and annotate stored video, audio, sensor telemetry, and spatial data captured by the system held within the learning engine and giving the user the ability to review customized recorded evaluation and augmented feedback once the procedure is finalized in an archived mode.

Certain aspects and embodiments of the present disclosure including combinations of sensing hardware and AI-based learning can be applied to other environments within healthcare facilities and perhaps in other critical environments, such as in manufacturing or restaurant environments where proper sanitation is of concern.

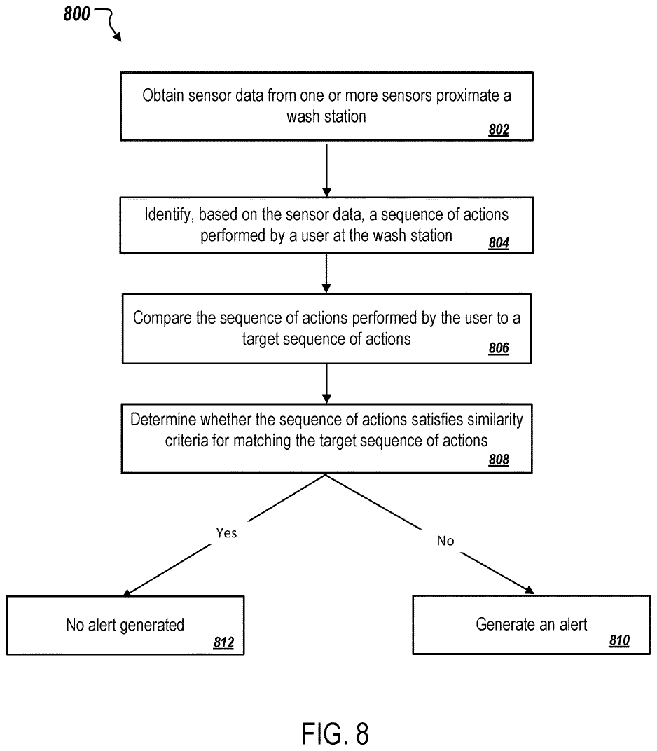

FIG. 8 depicts a flowchart of an example process 800 for monitoring a washing station. Process 800 can be executed by one or more computing systems including, e.g., the systems 100, 300, described above.

The system obtains sensor data from one or more sensors proximate a wash station (802). For example, the system can obtain one or more video feeds from stereographic cameras mounted near the wash station. In some examples, the system can identify (e.g., through object detection algorithms), objects at in the wash station 312 such as the sink 314, faucet 316, sponge, nail pick, water, soap, and iodine sponge. In some examples, the sensor data includes video images generated by a thermal imaging camera.

Sensor data can also include, for example, thermal images from the thermal imaging camera 304, temperature from the temperature sensor 306, water flow data from the water flow sensor 308, and audio data from the microphone 310.

The system identifies, based on the sensor data, a sequence of actions performed by a user at the wash station (804). For example, the system can employ object detection and tracking algorithms to identify body parts of humans within the video data and to track movements of the body parts captured by the video data. In some examples, the system can process the video data to generate a skeletal poise model of a human's body parts, such as a skeletal poise model of a human's hands being washed at the wash station. The system uses the skeletal poise models to identify wash actions performed by the body parts (e.g., rinsing, scrubbing, cleaning fingernails, drying). In some examples, identifying the sequence of actions includes determining a duration of each action. For example, the system can determine that a user scrubbed their left forearm for a time duration of forty-five seconds. In some examples, identifying the sequence of actions includes determining an ordering of the sequence of actions performed by the user. For example, the system can determine that the user scrubbed their left forearm before scrubbing their left wrist.

The system compares the identified sequence of actions performed by the user to a target sequence of actions (806). The target sequence of actions can be a sequence of actions associated with adequate surgical handwashing techniques. The target sequence of actions can include handwashing steps executed on a user's hands and forearms. The target sequence of actions can include, for example, removing jewelry, rinsing the hands and forearms with water, cleaning fingernails with a nail pick, scrubbing the hands and forearms with an iodine sponge, passing the hands and forearms through running water in a forward direction, washing the hands and forearms in a direction from the fingertips towards the elbow; and drying the hands and forearms with a towel. In some examples, the target sequence of actions includes maintaining parts of a user's body in particular positions. For example, the target sequence of actions can include maintaining the user's elbows bent at or near a right angle and maintaining the user's hands above the level of the elbows. The system can compare the position of the user's hands and elbows to a target positioning of the user's hands and elbows.

In some examples, the target sequence of actions includes washing a part of a user's body using water having a temperature within a designated range of temperatures.

Comparing the sequence of actions performed at the wash station to the target sequence of actions can include comparing the temperature of water used to wash the part of the user's body to the designated range of temperatures. In some examples, the temperature of the water can be determine using temperature data from a temperature sensor 306 (e.g., a thermometer). In some examples, the temperature of the water can be determined using thermal imaging data from the thermal imaging camera 304. For example, the designated range of temperatures for washing hands may be between one hundred degrees Fahrenheit and one hundred eight degrees Fahrenheit. The system can compare the temperature of water used to wash the user's hands with the designated range.

In some examples, comparing the sequence of actions performed at the wash station to a target sequence of actions includes comparing the duration of each action in the hand washing sequence to a target duration for the respective action in the target sequence of actions. For example, the target sequence of actions can include washing each forearm for at least one minute. The system can compare the time duration for which the user washes the left forearm (e.g., forty-five seconds) to the time duration of at least one minute.

In some examples, comparing the sequence of actions performed at the wash station to a target sequence of actions includes comparing the ordering of the sequence actions performed by the user to the order of the target sequence of actions. For example, the target sequence of actions can include an ordering of scrubbing the left wrist before scrubbing the left forearm. The system can compare the ordering of actions performed by the user (e.g., scrubbing the left forearm before scrubbing the left wrist) to the target ordering.

In some examples, the target sequence of actions includes introducing objects into the wash station, and comparing the sequence of actions performed at the wash station to the target sequence of actions can include comparing an object introduced into the wash station to the objects introduced in the target sequence of actions. For example, the target sequence of actions can include introducing a nail pick into the wash station. The user may introduce a nail pick into the wash station, and the system can determine that the introduced object matches the object in the target sequence of actions. The user may introduce a pencil into the wash station, and the system can determine that the introduced object does not match the object in the target sequence of actions.

The system determines whether the sequence of actions satisfies similarity criteria for matching the target sequence of actions (808). In some examples, determining whether the sequence of actions performed at the wash station satisfies similarity criteria for matching the target sequence of actions includes determining whether the temperature of the water used to wash the part of the user's body is within the designated range of temperatures. For example, for a designated range of temperatures of between one hundred degrees Fahrenheit and one hundred eight degrees Fahrenheit, and a temperature of the water of ninety-five degrees Fahrenheit, the system can determine that the temperature of the water used to wash the part of the user's body is not within the designated range of temperatures and, therefore, the sequence of actions performed by the user does not satisfy similarity criteria. In some examples, instead of a range of temperatures, the target temperature can include a designated temperature and a margin of error. For example, the target temperature can be one hundred five degree Fahrenheit plus or minus five degrees Fahrenheit.

In some examples, determining whether the sequence of actions performed at the wash station satisfies similarity criteria for matching the target sequence of actions includes determining whether a duration of at least one action of the sequence of actions performed by the user is less than a duration of a corresponding action of the target sequence of actions. For example, for a target duration of one minute for washing the left forearm, and a performed duration of forty-five seconds of washing the left forearm, the system can determine that the time duration of washing is less than the target time duration, and, therefore, the sequence of actions performed by the user does not satisfy similarity criteria. In some examples, the target time duration for an action in the target sequence can include a designated time duration and a margin of error. For example, the target time duration for a particular action (e.g., washing the left forearm) can be one minute plus or minus ten seconds.

In some examples, determining whether the sequence of actions performed at the wash station satisfies similarity criteria for matching the target sequence of actions includes determining whether the ordering of the sequence of actions performed by the user is different from the ordering of the target sequence of actions. For example, a target ordering can include washing a wrist prior to forearm, and if it is the determined the user washed the user's forearm prior to washing the user's wrist, the system can determine that the order of actions performed by the user is different from the target ordering and, therefore, the sequence of actions performed by the user does not satisfy the similarity criteria. In some examples, multiple different target orderings can be permissible. For example, a target ordering can include washing the fingers from outside to inside or from inside to outside.

In some examples, determining whether the sequence of actions performed at the wash station satisfies similarity criteria for matching the target sequence of actions includes determining whether an object introduced into the wash station does not match objects introduced in the target sequence of actions. For example, object introduced in the target sequence of actions can include a nail pick and a sponge. For an introduced object of a pencil, the system can determine that the introduced object does not match objects in the target sequence of actions, and, therefore, the sequence of actions performed by the user does not satisfy similarity criteria.

In response to determining that the sequence of actions performed by the user does not satisfy the similarity criteria, the system generates an alert (810). For instance, the alert can be audible (e.g., an alarm), visual (e.g., text presented on a display, illuminated light), or both. In some examples, the alert is overlaid on a display of the video. In some examples, the alert is presented on a display that is visible to the user performing the washing actions. In some examples, the alert is presented on a display that is visible to another user. The alert can indicate that actions performed at the wash station do not match target actions. For example, the alert can indicate that the user skipped a washing step, the user performed washing steps out of order, the user introduced an impermissible object into the washing station, the user failed to remove an impermissible object from the washing, station, a duration of a washing step is less than a minimum permissible duration, or any combination thereof. In some implementation, no alert is generated if the system determines that the sequence of actions performed by the user satisfies the similarity criteria (812).

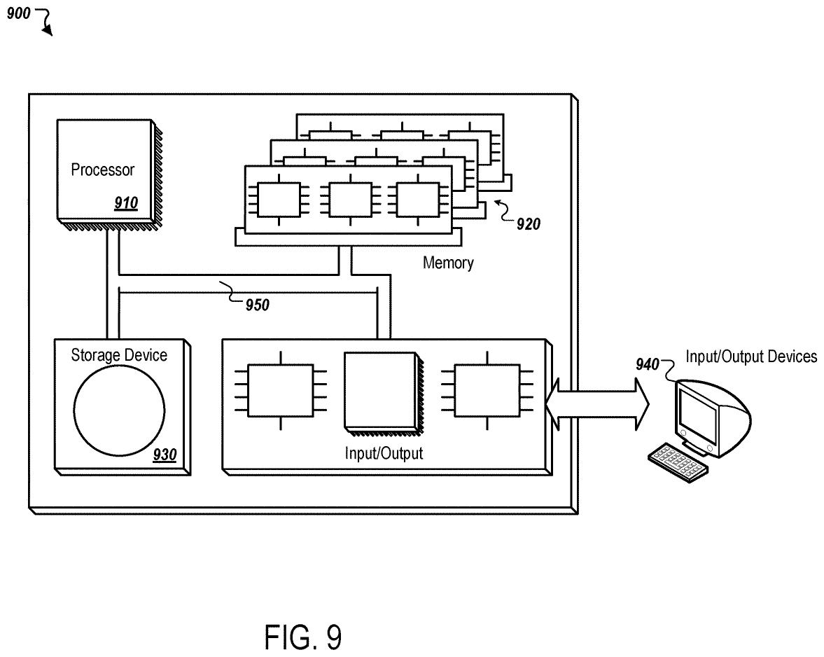

FIG. 9 is a schematic diagram of a computer system 900. The system 900 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 100, 300) and their structural equivalents, or in combinations of one or more of them. The system 900 is intended to include various forms of digital computers, such as laptops, desktops, workstations, personal digital assistants, servers, blade servers, mainframes, and other appropriate computers, including vehicles installed on base units or pod units of modular vehicles. The system 900 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 900 includes a processor 910, a memory 920, a storage device 930, and an input/output device 940. Each of the components 910, 920, 930, and 940 are interconnected using a system bus 950. The processor 910 is capable of processing instructions for execution within the system 900. The processor may be designed using any of a number of architectures. For example, the processor 910 may be a CISC (Complex Instruction Set Computers) processor, a RISC (Reduced Instruction Set Computer) processor, or a MISC (Minimal Instruction Set Computer) processor.

In one implementation, the processor 910 is a single-threaded processor. In another implementation, the processor 910 is a multi-threaded processor. The processor 910 is capable of processing instructions stored in the memory 920 or on the storage device 930 to display graphical information for a user interface on the input/output device 940.

The memory 920 stores information within the system 900. In one implementation, the memory 920 is a computer-readable medium. In one implementation, the memory 920 is a volatile memory unit. In another implementation, the memory 920 is a non-volatile memory unit.

The storage device 930 is capable of providing mass storage for the system 900. In one implementation, the storage device 930 is a computer-readable medium. In various different implementations, the storage device 930 may be a floppy disk device, a hard disk device, an optical disk device, or a tape device.

The input/output device 940 provides input/output operations for the system 900. In one implementation, the input/output device 940 includes a keyboard and/or pointing device. In another implementation, the input/output device 940 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

1. A computer-implemented method for monitoring a wash station, comprising:
obtaining sensor data from one or more sensors proximate the wash station;
identifying, based on the sensor data, a sequence of actions performed by a user at the wash station;
comparing the sequence of actions performed by the user at the wash station to a target sequence of actions;
determining whether the sequence of actions performed by the user at the wash station satisfies similarity criteria for matching the target sequence of actions; and
in response to determining that the sequence of actions performed by the user at the wash station does not satisfy the similarity criteria, generating an alert for presentation on a display device.

2. The method of claim 1, wherein:
the sensor data comprises video data; and
identifying, based on the sensor data, the sequence of actions performed by a user at the wash station comprises:
identifying a part of a human body in the video data;
generating, for the part of the human body, a skeletal poise model; and
using the skeletal poise model to track movements of the part of the human body in the video data, wherein the sequence of actions performed by the user at the wash station includes a sequence of movements of the part of the human body.

3. The method of claim 1, wherein:
identifying, based on the sensor data, the sequence of actions performed by the user at the wash station comprises determining, for each action of the sequence of actions, a duration of the action, and
comparing the sequence of actions performed by the user at the wash station to a target sequence of actions comprises comparing the duration of each action of the sequence of actions performed by the user at the wash station to a duration of each action of the target sequence of actions.

4. The method of claim 3, wherein determining that the sequence of actions performed by the user at the wash station does not satisfy similarity criteria for matching the target sequence of actions comprises determining that a duration of at least one action of the sequence of actions performed by the user at the wash station is less than a duration of a corresponding action of the target sequence of actions.

5. The method of claim 1, wherein:
identifying, based on the sensor data, the sequence of actions performed by the user at the wash station comprises determining, based on the sensor data, an ordering of the sequence of actions performed, and
comparing the sequence of actions performed by the user at the wash station to a target sequence of actions comprises comparing the ordering of the sequence of actions to an ordering of the target sequence of actions.

6. The method of claim 5, wherein determining that the sequence of actions performed by the user at the wash station does not satisfy similarity criteria for matching the target sequence of actions comprises determining that the ordering of the sequence of actions performed by the user at the wash station is different from the ordering of the target sequence of actions.

7. The method of claim 1, wherein the target sequence of actions includes washing actions executed on a user's hands and forearms, including one or more of:
removing jewelry;
rinsing the user's hands and forearms with water;
cleaning fingernails with a nail pick;
scrubbing the user's hands and forearms with an iodine sponge;
passing the user's hands and forearms through running water in a forward direction;
washing the user's hands and forearms in a direction from the fingertips towards the elbow; and
drying the user's hands and forearms with a towel.

8. The method of claim 1, wherein the target sequence of actions includes maintaining positions of parts of a user's body, including one or more of:
maintaining the user's elbows bent at or near a right angle; and
maintaining the user's hands above the level of the elbows.

9. The method of claim 1, wherein:
the target sequence of actions includes introducing one or more objects into the wash station, and
comparing the sequence of actions performed by the user at the wash station to a target sequence of actions comprises comparing an object introduced into the wash station to the one or more objects introduced in the target sequence of actions.

10. The method of claim 9, wherein determining that the sequence of actions performed by the user at the wash station does not satisfy similarity criteria for matching the target sequence of actions comprises determining that the object introduced into the wash station does not match the one or more objects introduced in the target sequence of actions.

11. The method of claim 1, wherein:
the target sequence of actions includes washing a part of a user's body using water having a temperature within a designated range of temperatures, and
comparing the sequence of actions performed by the user at the wash station to a target sequence of actions comprises comparing the temperature of water used to wash the part of the user's body to the designated range of temperatures.

12. The method of claim 11, wherein determining that the sequence of actions performed by the user at the wash station does not satisfy similarity criteria for matching the target sequence of actions comprises determining that the temperature of the water used to wash the part of the user's body is not within the designated range of temperatures.

13. The method of claim 1, wherein the sensor data comprises video images from at least one of a thermal imaging camera or a stereographic camera.

14. One or more non-transitory computer readable storage media storing instructions that, when executed by at least one processor, cause the at least one processor to perform operations comprising:
obtaining sensor data from one or more sensors proximate a wash station;
identifying, based on the sensor data, a sequence of actions performed by a user at the wash station;
comparing the sequence of actions performed by the user at the wash station to a target sequence of actions;
determining whether the sequence of actions performed by the user at the wash station satisfies similarity criteria for matching the target sequence of actions; and
in response to determining that the sequence of actions performed by the user at the wash station does not satisfy the similarity criteria, generating an alert for presentation on a display device.

15. A medical system, comprising:
one or more sensor devices configured to monitor a wash station;
a display device; and
at least one processor communicatively coupled with the sensor devices and the display device, the at least one processor being configured to perform operations comprising:
obtaining sensor data generated by the one or more sensor devices;
identifying, based on the sensor data, a sequence of actions performed by a user at the wash station;
comparing the sequence of actions performed by the user at the wash station to a target sequence of actions;
determining whether the sequence of actions performed by the user at the wash station satisfies similarity criteria for matching the target sequence of actions; and
in response to determining that the sequence of actions performed by the user at the wash station does not satisfy the similarity criteria, generating an alert for presentation on the display device.

16. The medical system of claim 15, wherein:
the sensor data comprises video data; and
identifying, based on the sensor data, the sequence of actions performed by a user at the wash station comprises:
identifying a part of a human body in the video data;
generating, for the part of the human body, a skeletal poise model; and
using the skeletal poise model to track movements of the part of the human body in the video data, wherein the sequence of actions performed by the user at the wash station includes a sequence of movements of the part of the human body.

17. The medical system of claim 15, wherein:
identifying, based on the sensor data, the sequence of actions performed by the user at the wash station comprises determining, for each action of the sequence of actions, a duration of the action, and
comparing the sequence of actions performed by the user at the wash station to a target sequence of actions comprises comparing the duration of each action of the sequence of actions performed by the user at the wash station to a duration of each action of the target sequence of actions.

18. (canceled)

19. The medical system of claim 15, wherein:
identifying, based on the sensor data, the sequence of actions performed by the user at the wash station comprises determining, based on the sensor data, an ordering of the sequence of actions performed, and
comparing the sequence of actions performed by the user at the wash station to a target sequence of actions comprises comparing the ordering of the sequence of actions to an ordering of the target sequence of actions.

20-22. (canceled)

23. The medical system of claim 15, wherein:
the target sequence of actions includes introducing one or more objects into the wash station, and
comparing the sequence of actions performed by the user at the wash station to a target sequence of actions comprises comparing an object introduced into the wash station to the one or more objects introduced in the target sequence of actions.

24. (canceled)

25. The medical system of claim 15, wherein:
the target sequence of actions includes washing a part of a user's body using water having a temperature within a designated range of temperatures, and
comparing the sequence of actions performed by the user at the wash station to a target sequence of actions comprises comparing the temperature of water used to wash the part of the user's body to the designated range of temperatures.

26-27. (canceled)
